(19) United States
(12) Patent Application Publication
(10) Pub. No.: US 2012/0301355 A1
(43) Pub. Date: Nov. 29, 2012

(54) Heatable Flange

(75) Inventors: Dirk Leutz, Ergoldsbach (DE); Matthias Weinzierl, Eching (DE); Jörg Zacharias, Koefering (DE); Torsten Runge, Straubing (DE)

(73) Assignee: KRONES AG, Neutraubling (DE)

(21) Appl. No.: 13/462,008

(22) Filed: May 2, 2012

(30) Foreign Application Priority Data
May 2, 2011 (DE) 10 2011 075 088.6

(51) Int. Cl. A61L 2/04; F16L 23/00; F16L 53/00; A61L 2/16

(52) U.S. Cl. 422/28; 422/1; 422/38; 285/405; 285/41

(57) ABSTRACT

A method and device for sterilizing a flange connection including a sealing seat used for establishing a connection between product-bearing pipelines, and where the sealing seat of the flange connection is treated by thermal energy input from the not product-bearing side.

### CROSS-REFERENCE TO RELATED APPLICATION

The present application claims the benefit of priority of German Application No. 102011075088.6, filed May 2, 2011. The entire text of the priority application is incorporated herein by reference in its entirety.

### FIELD OF THE DISCLOSURE

The disclosure relates to a method of sterilizing a flange connection comprising a sealing seat, and to a flange for carrying out the method.

In particular in the food industry, the sterility of product-bearing pipelines must be guaranteed to ensure a constant quality of the products. Therefore, the pipelines are usually treated with hot steam for an extended period of more than 20 minutes after their installation, after maintenance works or in case of product changes in the product-bearing region, that means inside the pipeline. The energy demand required for this, but in particular the energy loss arising in the process when the complete system is sterilized in this manner, is very high. Therefore, as a further possibility of reducing the germination index, chemical disinfection is often carried out. While the application of chemicals involves fewer costs and requires less time, chemical cleaning often involves the disadvantage that the space behind installed seals is often not reached by the disinfectant.

### SUMMARY OF THE DISCLOSURE

It is therefore one aspect of the disclosure to provide a method and a flange that permits the sterilization of the installed sealing seat with minimum energy consumption.

This is achieved according to the disclosure by a method wherein a flange connection comprising a sealing seat for establishing a connection between product-bearing pipelines is treated by thermal energy input from the not product-bearing side, that means from outside. This energy introduced from outside is suited for locally increasing the temperature in the region of the sealing seat. The thermal treatment of the sealing seat at a suited temperature over a sufficient period permits a local reduction of the number of germs and microorganisms in this region. In this manner, an energy-efficient sterilization of obstructed regions which are not reached during cleaning with chemicals, in particular with liquid disinfectants, can also be achieved.

To keep the description simple, only the killing effect with respect to germs will be described below, however, the thermal sterilization also refers to microorganisms, viruses, spores and the like.

The flange connection itself here consists of at least one flange according to the disclosure. In a flange connection between two product-bearing pipelines, the contact surfaces of the flanges contact each other and the seal cooperates with the sealing seat such that a tight connection is formed between the pipelines.

In the flange according to the disclosure, a channel is preferably provided in a region near the sealing seat through which energy input is permitted from the not product-bearing outer side.

In this method according to the disclosure, the sealing seat of the flange is sterilized in the obstructed region of the seal by an energy input being effected, preferably locally directed on the sealing seat from the not product-bearing side, which, as a temperature introduction of a sufficiently high temperature, has a germ-reducing effect. The heating essentially only concentrated on the sealing seat or the region obstructed by the seal involves only little energy consumption.

The thermal energy input is preferably effected by supplying a hot medium.

The hot medium preferably has a temperature of at least 120° C.

Preferably, steam or hot water is provided as hot medium.

In a further step, which can also be effected before the method according to the disclosure, the product-bearing region, that means the inner region of the pipeline which is in direct contact with the product, can also be treated with chemicals having a disinfecting effect, for example with liquid disinfectant. For this, the disinfectant acts upon the inner side of the product-bearing pipeline system. In this manner, the number of germs at the inner side of the pipes or at the tank walls is reduced and sterilized thereby. In combination with the thermal treatment of the flange connection according to the disclosure, a sterilization of the complete pipeline can be achieved thereby. These two cleaning steps can be carried out independent of each other, but preferably simultaneously.

In a preferred embodiment, at least one channel is provided at the flange according to the disclosure which comprises a first and a second channel opening and a channel center in-between. The first channel opening is preferably arranged at the upper side of the flange, that means at the side of the flange which lies opposite to the contact surface. The channel center spatially extends over a thin partition separated from it in a region near the sealing seat. The second channel opening is preferably arranged in the contact surface of the flange.

In a first variant, this second channel opening is arranged such that with a connection between a first and a second flange according to the disclosure, a continuous connecting channel is formed which causes a flush connection between the second channel opening of the first flange and the corresponding second channel opening of the second flange. In a second variant, the two channels are separated. Via the region of a first channel opening, a heating medium is then supplied to one of the two channels, and via the region of a second channel opening of the other channel, a heating medium is also supplied. A condensate formed in the first channel is discharged at a second channel opening of the first channel, whereas condensate formed in the second channel is discharged at a first channel opening of this second channel.

A hot medium is preferably conducted through this connection channel for sterilization. If, for example, hot steam or hot water is used as energy-bearing medium and supplied to the connection channel, the temperature will rise in particular in the region of the sealing seat. As a result of the heat conduction of the flange material, this temperature rise will also become effective in adjacent regions of the channel, though in particular at the sealing seat only separated by the thin partition. Due to the geometry of the channel or the connecting channel, respectively, the introduced thermal energy is concentrated at the sealing seat.

In another preferred embodiment, means for heating the sealing seat are arranged at the flange according to the disclosure. These heating means can be designed, for example, as a resistance wire, heating cord, strip heater, heated hose, or else as a Peltier element. These heating means thus permit, together with further elements, such as temperature sensors and voltage sources, both a temperature change preferably based on electric energy supply and a simple temperature control.

These heating means can be preferably introduced into one or several disposed channels. At least one of these channels can here also comprise a first channel opening which is preferably arranged at the upper side of the flange. The channel center extends in the region near the sealing seat. The second channel opening can also be arranged in the contact region. As an alternative, the channel can also end at any arbitrary point in the flange, so that there is no second channel opening. The heating means in the channel in this case preferably occupies the complete channel space. The electric supply lines for controlling or checking temperature can be supplied, for example, via the first channel opening.

The channels of the two flanges do not necessarily form a continuous connection channel in this variant.

In this embodiment of the flange, for the establishment of a flange connection it is sufficient to only design one flange according to the disclosure. If the flange connection consists of two flanges according to the disclosure, one common heating means can be operated, or the respective heating means can be operated each separately or together by means of a voltage source.

If the voltage adjustable at the voltage source of the heating means is increased, the introduced electric energy will propagate in the form of heat in the flange, in particular in the region of the sealing seat. In this manner, the region behind the installed seal can be selectively heated and thus sterilized.

In an additional cleaning step, the product-bearing region of the pipeline can also be cleaned with a chemical having a disinfecting effect. This cleaning step leads, in combination with the method according to the disclosure, to a complete sterilization of the total pipeline.

### BRIEF DESCRIPTION OF THE DRAWINGS

Further advantages and embodiments result from the enclosed drawings. In the drawings:

FIG. 1 shows a view of a pipe connection consisting of two flanges according to the disclosure;

FIG. 2a shows a schematic sectional view of the region A of FIG. 1 in a first embodiment of the flange according to the disclosure;

FIG. 2b shows a schematic sectional view of the region A of FIG. 1 in a second embodiment of the flange according to the disclosure;

FIG. 3 shows a schematic sectional view of the region A of FIG. 1 in a third embodiment of the flange according to the disclosure.

### DETAILED DESCRIPTION OF THE PREFERRED EMBODIMENT

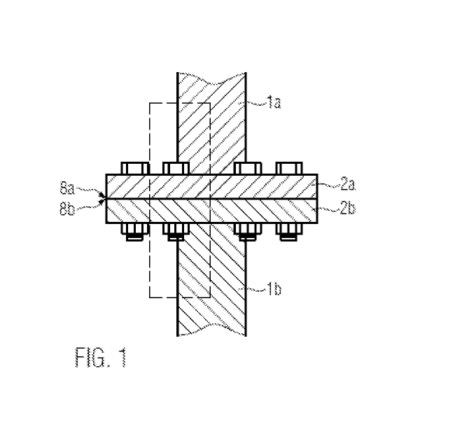

FIG. 1 shows two product-bearing pipelines which are interconnected via a flange connection. The pipeline system here comprises a first line region 1a with a first flange 2a and a second line region 1b with a second flange 2b. The connection between the flange and the line region can here be screwed, welded or established in any other way. The connection between the two flanges 2a, 2b, where in particular the contact surfaces 8a, 8b of the flanges are pressed against each other, is established, for example, via one or several screwing means.

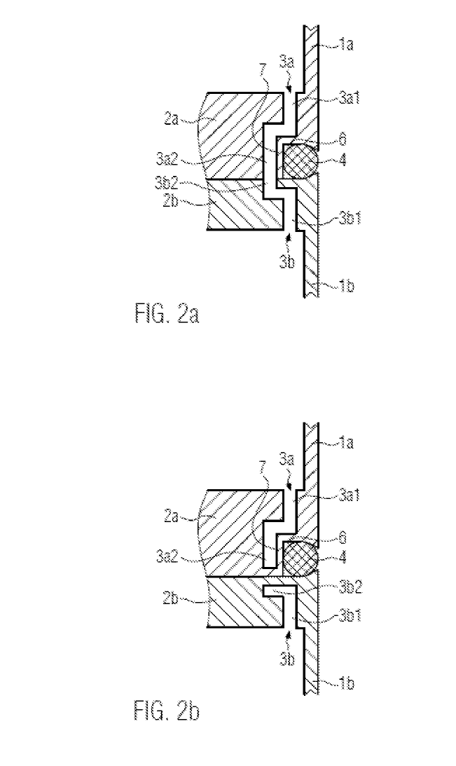

A first embodiment of the flange according to the disclosure or a flange connection consisting thereof is shown in FIG. 2a. Here, a sectional representation of the marked region A of FIG. 1 is shown. As indicated here, the sealing seat 6 and the seal 4 cooperate such that a tight connection between the pipelines 1a and 1b is formed. The seal 4 can here comprise, for example, the sealing material Viton, EPDM or PTFE.

In this embodiment, in the first flange 2a, a first channel 3a is provided, and in the second flange 2b, a second channel 3b is provided. Both channels extend such that the respective first channel opening 3a1, 3b1 is arranged in the not product-bearing external region at the upper side of the flange, and the respective second channel opening 3a2, 3b2 is arranged at the contact surfaces 8a, 8b between the flanges 2a, 2b. The channels extend in a region near the sealing seat 6.

In the region of the sealing seat, the connection channel extends as close as possible to the side of the seal facing away from the product. The sealing seat 6 itself is preferably separated from the channel 3a, 3b by a small partition 7. The obstructed region of the sealing seat, in particular the region behind the seal, is often not reached by chemical sterilization within the product-bearing line region.

The second channel openings 3a2, 3b2 at the contact surface 8a, 8b of the two flanges are here adjusted to each other such that a flush connection between the channel 3a and the channel 3b is formed. In this manner, a hot medium can be introduced, for example, into the first channel opening 3a1 of the first flange 2a and, after it has passed through the region close to the seal, exit again at the first channel opening 3b1 of the second flange 2b.

As a hot medium, steam is used here. Hot water could be also used, for example. This hot steam preferably gets through the first channel opening 3a1 of the first flange 2a into the connection channel connecting the two flanges. When it flows through the connection channel, in particular in the region near the seal, the steam emits energy to the surrounding material of the channel and heats it up. The heat transfer through the partition 7 to the adjacent sealing seat 6 permits to selectively locally heat this region. Here, a partition 7 as thin as possible is to be preferred to reduce sterilization time. During the cooling in connection with it, the steam condensates and the formed condensate exits from the connection channel downwards through the first channel opening 3b1 of the second flange 2b.

The exact channel extension of the channel 3a or the channel 3b can here be adapted to the medium flowing through them. In the represented case, hot steam is used for sterilization. Therefore, the extension selected for the channel 3b in the flange is designed such that the condensate formed during the passage through the channel can be discharged slightly downwards or drip off or be discharged for recycling it. Therefore, the first channel opening 3b1 in the second flange 2b is essentially oriented to be vertical, while the first channel opening 3a1 in the first flange 2a is preferably oriented away from the line region 1a to more easily supply steam.

A second embodiment of the flange according to the disclosure is also represented in FIG. 2b in a section. Here, different to the first embodiment, no continuous channel through the two flanges 2a and 2b is present, but the two channels 3a and 3b are separate from each other.

The first channel 3a is filled from the first channel opening 3a1 with a heating medium, for example steam, this heating medium then condensating as it flows through the channel 3a, so that the then formed condensate is removed again in the region of the second channel opening 3a2 of the first channel 3a.

The second channel 3b is filled in the region of the second channel openings 3b2 with the same or another heating medium. In the second channel 3b, too, condensation takes place, so that the condensate formed there is then removed in the region of the first channel openings 3b1 of the second channel 3b.

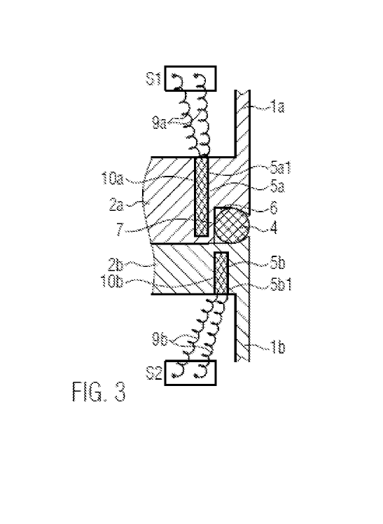

A third embodiment of the flange according to the disclosure is shown in FIG. 3 as a section. Analogous to the embodiment represented in FIG. 2, here, too, a first channel 5a in the first flange 2a and a second channel 5b in the second flange 2b are provided. Equally, however, one or several channels could be provided only in one flange of the flange connection. In this variant, one type of means each for heating the sealing seat 6 is provided, in this representation designed as a resistance wire 10a, 10b. Preferably, this resistance wire 10a, 10b is introduced into the respective channel 5a, 5b and completely occupies the channel 5a, 5b. Preferably, the resistance wire 10a, 10b is in contact with the channel wall at as many points as possible, so that an efficient transfer of thermal energy is possible. To improve this contact between the heating means and the channel wall, heat transfer pastes, heat transfer adhesives or the like can be used. Apart from the use of a resistance wire, a heating cord, a strip heater or a heated hose are conceivable.

In this embodiment, the course of the channel 5a, 5b is designed such that an extended section of the heating means is located near the sealing seat 6 but is spatially separated from it by a thin partition 7. The channel here only has a first channel opening 5a1, 5b1, the channel 5a, 5b ending in the flange, for example, in the form of a blind hole. Equally, however, a second channel opening could be provided for introducing or operating the heating means.

The electric lines 9a, 9b for operating the resistance wire 10a, 10b are supplied through the first channel opening 5a1, 5b1 and connected with one or several voltage sources S1, S2. By means of the applied voltage, the temperature of the resistance wire 10a, 10b can be determined.

If the voltage of the voltage source S1, S2 is increased, the temperature of the resistance wire 10a, 10b will also rise. The thermal conductivity of the surrounding flange material leads to a temperature rise of the region adjacent to the channel 5a, 5b, but in particular of the region near the sealing seat 6. The control of the applied voltage permits a selective temperature-control and sterilization of the installed region of the sealing seat 6.

As an alternative to the use of channels into which the heating means is inserted, for example, the heating means could be already incorporated during the manufacture of the flange. This could be, for example, a Peltier element or a heating mat which are arranged flatly at the contact surfaces 8a, 8b of the flanges 2a, 2b such that these heating means can thermally act upon the region of the sealing seat 6 and only their electric lines 9a, 9b are conducted to the outside via channels.

In the embodiments shown here, only the flange or the flange connection and the method of sterilizing the space behind the installed seal are described. Apart from this, the inner wall of the pipeline or the line regions 1a, 1b can be sterilized with a chemical having a disinfecting effect, for example with a disinfectant.

1. A method of sterilizing a flange connection, comprising using a sealing seat for establishing a connection between product-bearing pipelines, and treating the sealing seat of the flange connection by thermal energy input from the not product-bearing side.

2. The method according to claim 1, and effecting the thermal energy input by supplying a hot medium.

3. The method according to claim 2, and wherein the hot medium has a temperature of at least 120° C.

4. The method according to claim 2, and providing steam or hot water is provided as the hot medium.

5. The method according to claim 1, and effecting the thermal energy input by using means for heating.

6. The method according to claim 1, and disinfecting the product-bearing region of the pipeline by chemicals.

7. A flange for carrying out the method according to claim 1, comprises comprising at least one channel which extends in a region near the sealing seat and through which the energy input is effected from the not product-bearing side.

8. The flange according to claim 7, wherein the channel is designed such that it extends flush from the first to the second flange in the establishment of a flange connection consisting of a first and a second flange.

9. A flange for carrying out the method according to claim 1, and comprising means for heating provided in the flange.

10. The method according to claim 6, wherein the chemicals comprise disinfectants, electrochemically activated water, and a combination thereof.
